(19) United States
(12) Patent Application Publication
(10) Pub. No.: US 2024/0315615 A1
(43) Pub. Date: Sep. 26, 2024

(54) DETECTION ELEMENT AND A WEARABLE DEVICE OF BIOLOGICAL SUBCUTANEOUS FEATURE INCLUDING THE SAME

(71) Applicant: Lextar Electronics Corporation, Hsinchu (TW)

(72) Inventors: Kai-Hung CHENG, Hsinchu City (TW); Ku-Cheng LIN, Hsinchu City (TW); Chun-Min LIN, Hsinchu City (TW); Ke-Wei LIU, Hsinchu City (TW)

(21) Appl. No.: 18/610,147

(22) Filed: Mar. 19, 2024

(30) Foreign Application Priority Data
Mar. 20, 2023 (TW) 112110180

(51) Int. Cl. A61B 5/1455

(52) U.S. Cl. CPC A61B 5/14552

(57) ABSTRACT

A detection element of biological subcutaneous features and a wearable device thereof are provided. The element for detecting biological subcutaneous features has a substrate, a light-detecting semiconductor chip, a grid structure, and a cover. The light-detecting semiconductor chip is located on the substrate for detecting red light or near-infrared light signals. The grid structure including a plurality of opaque light-absorbing blocking walls is located on the light-detecting semiconductor chip for blocking side light and increasing the proportion of near-vertical incident light. The cover is located on the grid structure and serves as a protection lid. The wearable device for detecting biological subcutaneous features has more than one light source of red light or near-infrared light and a plurality of detection elements. The arrangement of the opaque light-absorbing blocking walls that are parallel to each other are substantially parallel to the light-emitting directions of the light source.

### CROSS REFERENCE TO RELATED APPLICATIONS

This application claims priority of Taiwan Patent Application No. 112110180, filed on Mar. 20, 2023, the entirety of which is incorporated by reference herein.

### BACKGROUND OF THE DISCLOSURE

### Field of the Disclosure

The present disclosure relates to a detection element of biological subcutaneous features and a wearable device for detecting biological subcutaneous features, in particular to applications of blood oxygen detection and blood glucose detection. Biological subcutaneous features include signals from the dermis, subcutaneous tissue, muscle tissue, arteries, veins, capillaries, etc., which can be used to estimate biological vital features data such as blood oxygen and blood glucose.

### Description of the Related Art

With the advancement of technology, the use of optoelectronic devices for non-invasive detection of vital signs has become increasingly convenient and important. Various aspects, such as heartbeat, blood oxygen, and blood glucose, have been extensively researched. Even parameters like blood pressure and blood flow are subjects of ongoing scientific investigation. The primary driver behind this trend is the aging population, leading to the emergence of various lifestyle-related diseases such as hypertension and heart disease. Early detection is crucial for early treatment in these cases. On the other hand, numerous incurable diseases, such as diabetes and kidney disease, continue to afflict many patients. Consequently, there is an urgent demand for a variety of affordable, reliable, safe, and painless wearable devices for non-invasive detection of subcutaneous biological features. This demand has intensified, especially after the COVID-19 pandemic, with a pressing need for blood oxygen monitoring.

Blood oxygen detectors mainly use the Beer-Lambert Law in spectrophotometry. When light of a specific wavelength passes through a certain solution (the solution is composed of a solute that absorbs this light and a solvent that does not absorb this light), its light absorbance (A) is proportional to the absorption coefficient (α), light path length (l) and concentration (c). That is, A=α·l·c.

Currently, the mainstream design of blood oxygen detectors is to combine the design of light-emitting diodes with different emission wavelengths, light-detecting element and various algorithms to achieve a non-invasive detection method of detecting biological vital features. The tissues of the fingers, toes, or earlobes in the human body are thin and full of capillaries, so they are suitable for penetrating designs. On the contrary, the wrists, forehead, and torso are too thick, so only reflective designs can be used.

### BRIEF SUMMARY OF THE DISCLOSURE

The present disclosure provides a detection element of biological subcutaneous features and a wearable device for detecting biological subcutaneous features. The detection element of biological subcutaneous features has a substrate, a light-detecting semiconductor chip, a grid structure, and a cover. The light-detecting semiconductor chip is located on the substrate and is used to detect red light or near-infrared light signals. The grid structure is located on the light-detecting semiconductor chip and has a plurality of opaque blocking walls that are parallel to each other to block side light and increase the proportion of near-vertical incident light. The cover is located between or on the grid structure to protect the light incident surface of the element. A wearable device for detecting biological subcutaneous features has more than one light source of red light or near-infrared light, and a plurality of element for detecting biological subcutaneous features. One detection element of biological subcutaneous features has a grid structure, which has a plurality of opaque blocking walls that are parallel to each other. The arrangement direction of the opaque blocking walls is roughly parallel to the emission direction of the light emitted by the light source.

Detailed description is provided in the following embodiments with reference to the accompanying drawings.

### BRIEF DESCRIPTION OF THE DRAWINGS

FIG. 1 shows the absorption rates of hypoxic hemoglobin and oxyhemoglobin in different light wavelength ranges.

FIG. 2 shows the design structure of conventional non-invasive light-detecting of biological subcutaneous features.

FIG. 3 shows the change of light absorbance of blood with time and arterial pulse.

FIG. 4 is a top view and a side cross-sectional view of the manufacturing method of a light-detecting element according to an embodiment of the present disclosure: (a) with the black adhesive material coated on the light-detecting wafer (b) a grid structure formed after etching process (c) a cover formed by filling with silicone.

FIG. 5 is a perspective view of four detection elements of biological subcutaneous features according to an embodiment of the present disclosure: (a) a flip-chip light-detecting semiconductor chip and a silicone cover, (b) a flip-chip light-detecting semiconductor chip without a cover, (c) a vertical type light-detecting semiconductor chip and a glass cover, (d) a face-up type light-detecting semiconductor chip and a glass cover.

FIG. 6 is a top view and side cross-sectional of four detection elements of biological subcutaneous features: (a) without the grid structure, (b) an equally spaced grid structure, (c) a gradually sparse grid structure, (d) a gradually dense grid structure.

FIG. 7 shows four wearable devices for detecting biological subcutaneous features: (a) no grid structure (b) equally spaced grid structure (c) gradually sparse grid structure (d) gradually dense grid structure, for detecting biological subcutaneous features. Each wearable device is accompanied by a distribution diagram of light intensity corresponding to a 660 nm light source.

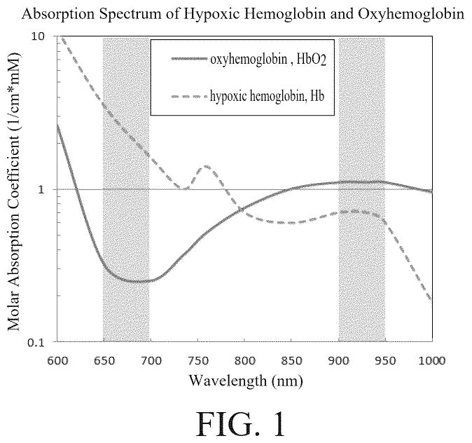

### DETAILED DESCRIPTION OF THE DISCLOSURE

The following describes the package structure and the method of forming the same of the embodiment of the present disclosure. It should be understood that the following description provides many different embodiments for implementing different aspects of some embodiments of the present disclosure. The specific elements and arrangements described below are only used to briefly and clearly describe some embodiments of the present disclosure. Of course, these are only examples and not limitations of the present disclosure. In addition, similar and/or corresponding element symbols may be used to identify similar and/or corresponding elements in different embodiments to clearly describe the present disclosure. However, the use of these similar and/or corresponding element symbols is only for the purpose of simply and clearly describing some embodiments of the present disclosure, and does not imply any correlation between the different embodiments and/or structures discussed.

Embodiments of the present disclosure provide a detection element of biological subcutaneous features and a wearable device including the same.

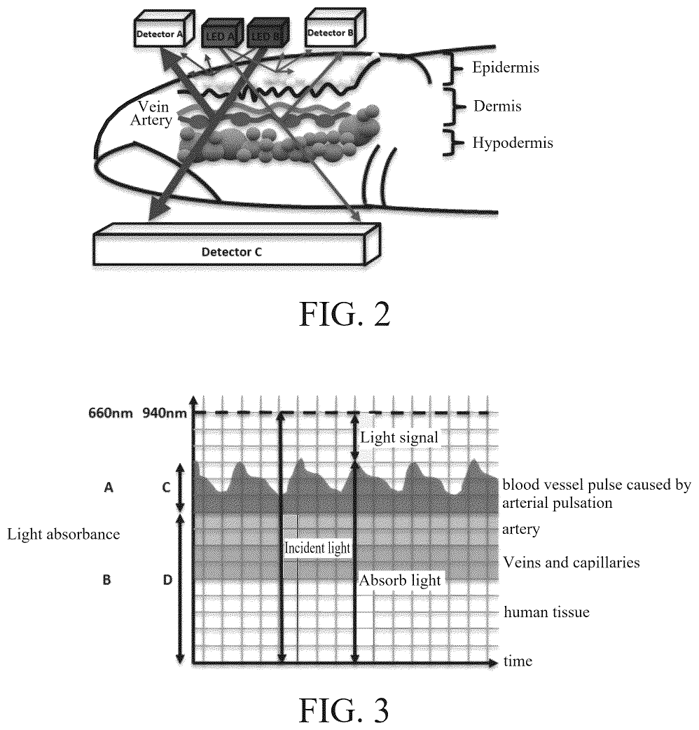

According to some embodiments of the present disclosure, a package structure is provided. The package structure has a substrate, a light-detecting semiconductor chip, a grid structure, and a cover. The light-detecting semiconductor chip is located on the substrate and can detect red light or near-infrared light signals. The grid structure is located on the light-detecting semiconductor chip to block side light and increase the proportion of near-vertical incident light. The cover is located between or on the grid structure and serves as a transparent light incident surface that protects the elements. In some embodiments of the present disclosure, the light-detecting semiconductor chip may be made of silicon-based semiconductor material, such as silicon semiconductor or silicon germanium semiconductor material. The light-detecting semiconductor chip may also be made of III-V semiconductor material, such as indium gallium arsenide semiconductor or indium phosphide semiconductor material. In some embodiments of the present disclosure, a reflective design structure is adopted as shown in FIG. 2. The LED light source A and LED light source B are light-emitting diodes with wavelengths of 660 nm and 940 nm respectively. The light-detecting package structure (Detector A, Detector B) is disposed on the same side of the object to be measured. This light-detecting package structure uses some embodiments of the present disclosure. The size and height of the opening of the grid structure on this package structure depend on the distance between the LED light source and the light-detecting semiconductor chips. When the distance between the light source and the light-detecting semiconductor chip is closer, the opening angle required by the structure can be smaller. On the contrary, when the distance is farther, the opening angle required by the structure should be larger to effectively block ambient light from entering the light-detecting semiconductor chip, ensuring that the light received by the light-detecting semiconductor chip is light from blood vessels and blood, thereby improving the accuracy of the signal.

The grid structure is composed of a plurality of opaque blocking walls that are parallel to each other, with an aspect ratio ranging from 1:1 to 100:1. For example, a plurality of opaque blocking walls that are parallel to each other, have a height of 100 μm to 1000 μm, a width of 20 μm to 100 μm, and a spacing of 50 μm to 600 μm. The grid structure may be fabricated directly on the light-detecting semiconductor chip using molding, cutting or photolithography.

The grid structure may also be pre-formed on a temporary substrate and then attached onto the light-detecting semiconductor chip using an adhesive material, or affixed onto the light-detecting semiconductor chip with a clasp.

The grid structure is made of opaque materials such as black resin, black silicone, and silicon substrate. The plurality of opaque blocking walls of the grid structure that are parallel to each other can be arranged with equal spacing or with gradually dense spacing. In some embodiments of the present disclosure, the cover may be made of resin, silicone, glass, or other materials that are substantially transparent to red light or near-infrared light, and may be directly filled or covered with the grid structure. The cover may also be completely covered around the light-detecting semiconductor chip and over the substrate, so that the 660 nm or 940 nm light emitted by the LED light source enters the human skin and is reflected back by the blood vessels, and then passes through the transparent cover to reach the light-detecting semiconductor chip. The opaque blocking walls of the grid structure may inhibit the absorption of part of the epidermal scattering that does not pass through blood vessels.

In some embodiments of the present disclosure, the light-detecting semiconductor chip that is used has three forms, namely flip-chip type, vertical type, and the face-up type. Above the light-detecting semiconductor chip is the one with the p-n junction or the p-i-n junction. Below the light-detecting semiconductor chip is the semiconductor substrate, which is relatively far away from the p-n junction or the p-i-n junction. Among them, in the design and production of flip-chip type light-detecting semiconductor chips, the light incident surface is designed above the chip, and both the positive and negative electrodes are made below the chip to maximize the light incident area of the chip. The package substrate is designed with two pads corresponding to the size and spacing of the two electrodes of the chip, which facilitates die-bonding with tin paste and electrically connect the positive and negative electrodes respectively. In the design and production of vertical type light-detecting semiconductor chips, the light incident surface is designed above the chip, one of the positive/negative electrodes is made below the chip, and the other electrode is made above the chip to form a wiring area. Its matching package substrate has a soldering pad larger than the chip area to facilitate die-bonding with tin paste and electrically connect with one of the positive/negative electrodes, and the electrode wiring area above it is electrically connected to another pad on the substrate by wiring. During the design and production of the face-up type light-detecting semiconductor chips, the light incident surface is designed above the chip, and the positive and negative electrodes are made above the chip to form two wiring areas respectively. The matching package substrate has a die-bond area that is larger than the chip area to facilitate die-bonding with glue or tin paste on the substrate. The positive electrode wiring area and the negative electrode wiring area are electrically connected to the two soldering pads on the substrate by wire bonding.

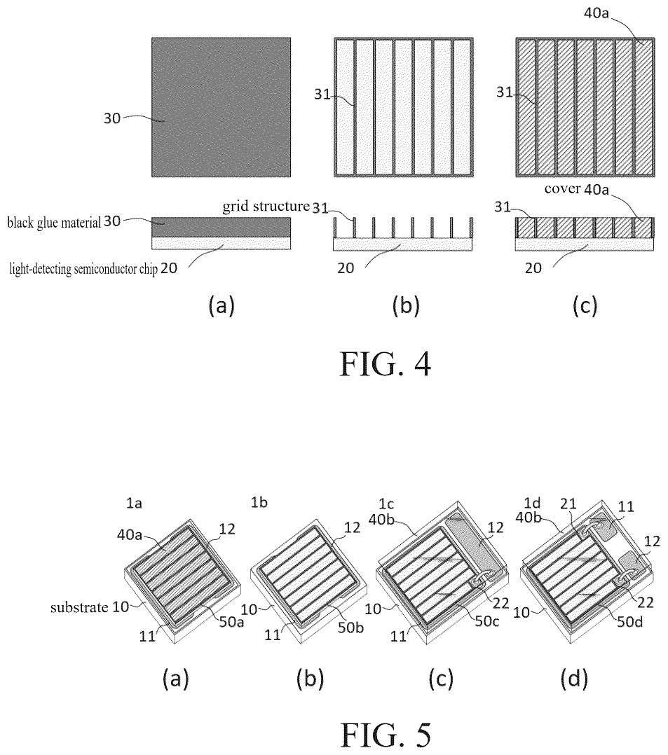

In some embodiments of the present disclosure, as shown in FIGS. 4 (a), (b), and (c), the carbon-based black powder is first added to silicone to form a black glue material. The black glue material is evenly coated on the flip-chip type light-detecting semiconductor chip 20 to form a black glue layer 30, and then the black glue layer 30 is etched to form multiple parallel grid structures 31. The grid structure 31 is filled with transparent silicone to form a cover 40a, and then the wafer is cut into light-detecting element 50a. The light-detecting element 50a is die-bonded and electrically connected to the package substrate 10, as shown in FIG. 5(a). The positive electrode below the chip is fixed and electrically connected to the positive electrode pad 11 of the substrate using tin paste. The negative electrode below the chip is fixed and electrically connected to the negative electrode pad 12 of the substrate using tin paste. Thus, a detection element of biological subcutaneous features 1a is completed.

In some embodiments of the present disclosure, a carbon-based black adhesive material is first made into a black adhesive layer 30 on a temporary substrate, and then cut or etched to form a grid structure 31 with multiple parallel grids. After filling the grid structure 31 with transparent silicone to form the cover 40a, the grid structure 31 and the cover 40a on the temporary substrate are directly attached to the flip-chip type light-detecting semiconductor chip 20. Then, the temporary substrate is removed and the cover 40a and the grid structure 31 are remained on the flip-chip type light-detecting semiconductor chip 20. After removing the temporary substrate, the wafer is cut into light-detecting element 50a, and the light-detecting element 50a is die-bonded and electrically connected to the package substrate 10, where the positive electrode below the chip is fixed and electrically connected to the positive electrode pads 11 of the substrate using tin paste. The negative electrode below the chip is fixed and electrically connected to the negative electrode pads 12 of the substrate using tin paste, as shown in FIG. 5(a). Thus, a detection element of biological subcutaneous features 1a is completed.

In some embodiments of the present disclosure, the light-detecting semiconductor chip 20 is first cut into light-detecting semiconductor chips 20a, and then a carbon-based black glue is made into a black glue layer 30 on the temporary substrate. It is made into multiple parallel grid structures 31 through cutting technology or etching technology, and then the grid structures 31 are filled with transparent silicone to directly form the cover 40a. The grid structure 31 and the cover 40a are then cut into thin slice units with the same size as the light-detecting semiconductor chip 20a through cutting technology. Then, the thin slice units are peeled off from the temporary substrate and attached to the light-detecting semiconductor chip 20a by one-to-one bonding or clasping to form the light-detecting element 50a. Then, the light-detecting element 50a is die-bonded and electrically connected to the package substrate 10. The positive electrode below the chip is fixed and electrically connected to the positive electrode pad 11 of the substrate using tin paste. The negative electrode below the chip is fixed and electrically connected to the negative electrode pads 12 of the substrate using tin paste, as shown in FIG. 5(a). Thus, a detection element of biological subcutaneous features 1a is completed.

In some embodiments of the present disclosure, as shown in FIGS. 4 (a) and (b), the carbon-based black powder is first added to silicone to form a black glue material, which is evenly coated on the flip-chip type light-detecting semiconductor chip 20 to form a black glue layer 30. Then, a plurality of parallel grid structures 31 are formed by etching the black glue layer 30. Then, the step of filling the grid structure 31 with transparent silicone to form the cover 40a is omitted, and the wafer with the grid structure 31 is directly cut into a light-detecting element 50b, as shown in FIG. 5(b). The light-detecting element 50b is die-bonded and electrically connected to the package substrate 10. The positive electrode below the chip is fixed and electrically connected to the positive electrode pad 11 of the substrate using tin paste. The negative electrode below the chip is fixed and electrically connected to the negative electrode pads 12 of the substrate using tin paste. Thus, a detection elements of biological subcutaneous features 1b is completed.

In some embodiments of the present disclosure, the vertical type and the face-up type light-detecting semiconductor chip 20 may be used according to the aforementioned implementation. Carbon-based black powder is first added to silicone to form a black glue material. The black glue material is evenly coated on the vertical type and the face-up type light-detecting semiconductor chip 20 to form a black glue layer 30. A plurality of parallel grid structures 31 are formed above the chip by etching the black adhesive layer 30. Also, the front side of the chip is etched to expose the reserved wiring areas 21/22 to facilitate subsequent electrical connections. Then, the step of filling the grid structure 31 with transparent silicone to form the cover 40a is omitted, and the wafer with the grid structure 31 is directly cut into the vertical type and the face-up type light-detecting elements 50c/50d, as shown in FIG. 5(c). The positive electrode of the vertical type light-detecting element 50c is fixed and electrically connected to the positive electrode pad 11 of the substrate using tin paste. The negative electrode 22 is electrically connected to the negative electrode pad 12 of the substrate by wire bonding. As shown in FIG. 5(d), the positive electrode 21 of the face-up type light-detecting element 50d is electrically connected to the positive electrode pad 11 of the substrate by wire bonding. The negative electrode 22 is electrically connected to the negative electrode pad 12 of the substrate by wire bonding. In some embodiments of the present disclosure, for designs that require wiring or do not have a transparent silicone cover 40a, a cover made of a material that is substantially transparent to red light or near-infrared light may be chosen. In this way, both the light-detecting element and the metal wire may be protected from damage caused by external force. As shown in FIG. 5(c) and FIG. 5(d), a transparent glass cover 40b is used. The transparent glass cover 40b is placed on the grid structure 31 and the substrate 10, thereby completing the detection elements of biological subcutaneous features 1c and 1d.

In some embodiments of the present disclosure, when viewed from a top view, the light-detecting element 50/50a/50b/50c/50d has a square structure, and the grid structure formed thereon has a height of 250 μm, a width of 30 μm, and a spacing of 210 μm. In some embodiments of the present disclosure, the structure of detection element of biological subcutaneous features may be rectangular, circular, or a polygon with more than four sides.

In some embodiments of the present disclosure, the grid structure 31 may be composed of a plurality of opaque blocking walls that are parallel to each other, and the spacing of the blocking walls varies. As a control group, as shown in FIG. 6(a), the traditional detection element of biological subcutaneous features 1ref is a structure without grid. As shown in FIGS. 6(b), 6(c), and 6(d), the other three detection elements of biological subcutaneous features 1a1/1a2/1a3 fabricated according to the technology in the present disclosure, and respectively have equally spaced grid structure 31b shown in FIG. 6(b), gradually sparse grid structure 31c shown in FIG. 6(c), and gradually dense grid structure 31d shown in FIG. 6(d). Among them, the grid spacing of the gradually sparse grid structure 31c increases as the distance from the center point of the surface of the top view of the chip decreases, while the grid spacing of the gradually dense grid structure 31d decreases as the distance from the center point of the surface of the top view of the chip decreases.

According to some embodiments of the present disclosure, a wearable device including the above-mentioned detection element of biological subcutaneous features is provided. The wearable device has more than one light source of red light or/and near-infrared light, and a plurality of detection elements of biological subcutaneous features. One detection element of biological subcutaneous features has a grid structure of a plurality of opaque blocking walls that are parallel to each other. The arrangement direction of the opaque blocking walls is not perpendicular to the direction of light emission from the light source. In another embodiment of the present disclosure, the arrangement direction of the opaque blocking walls is substantially parallel to the direction of light emission from the light source. In some embodiments of the present disclosure, the plurality of detection elements of biological subcutaneous features on the wearable device for detecting biological subcutaneous features surrounds a light source of red light or/and near-infrared light. In addition, the plurality of detection elements of biological subcutaneous features on the wearable device for detecting biological subcutaneous features is generally arranged with the light source as the center and radiating outward. Therefore, the arrangement direction of the opaque blocking walls of the grid structure of the plurality of detection elements of biological subcutaneous features is parallel to the light-emission direction of the light source. In some embodiments of the present disclosure, the wearable device that detects biological subcutaneous features may be a watch, an oximeter, or a blood glucose meter.

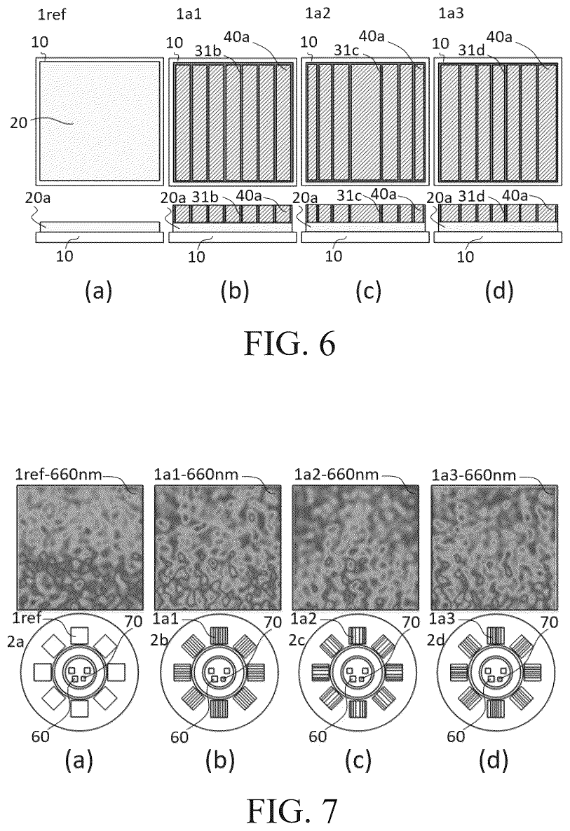

As shown in FIG. 3, the signals received by elements that detect biological subcutaneous features include human tissue, veins, capillaries, arteries, and vascular pulses caused by arterial pulsation. Among them, the vascular pulses caused by arterial pulsation are called alternating current signal AC, which may fluctuate with time, while the sum of other signals, called the direct current signal DC, is relatively stable. The perfusion index PI (PI=AC/DC) calculated based on the AC/DC ratio helps to determine whether the measured oximetry and pulse rate are accurate. Generally, the PI value range that the oximeter can measure is from 0.02% to 30%. If the PI value is too low, such as less than 0.4%, the reading displayed by the oximeter may not be a reliable reference. By improving the element design and increasing the PI value, the accuracy of the data can be improved. In some embodiments of the present disclosure shown in FIGS. 7(a), (b), (c), and (d), eight elements from the four different features 1ref/1a1/1a2/1a3 shown in FIG. 6 are used to detect biological subcutaneous features and are arranged around the LED light sources 60 with a wavelength of 660 nm and LED light sources 70 with a wavelength of 940 nm. These elements respectively form four wearable devices 2a/2b/2c/2d equipped with detection elements of biological subcutaneous features. As shown in Table 1, considering the received light energy corresponding to the 660 nm and 940 nm light intensity distribution of the detection element of biological subcutaneous features, the total light intensity (DC+AC) of 1a1—equally spaced grid structure/1a2—gradually sparse grid structure/1a3—gradually dense grid structure is lower than that of 1ref—no grid structure. However, because the proportion of light AC hitting the artery increases, the PI value actually increases. Therefore, the grid structure design may effectively block stray light to increase the proportion of light signals from blood vessels and blood, thus improving the accuracy of data. According to the results of these embodiments, the perfusion index PI value of the structure with the grid is better than that of the traditional structure without the grid.

TABLE I

| | | | 1a1-equally | 1a2-gradually | 1a3-gradually |
|---|---|---|---|---|---|
| | | 1ref-no grid | spaced grid | sparse grid | dense grid |
| | Signal intensity | structure | structure | structure | structure |
| 600 nm | DC + AC | 112.81 | 45.95 | 50.22 | 46.81 |
| | DC | 103.49 | 41.63 | 45.63 | 42.4 |
| | AC | 9.32 | 4.32 | 4.59 | 4.41 |
| | PI = AC/DC | 9.0% | 10.4% | 10.1% | 10.4% |
| 940 nm | DC + AC | 113.02 | 47.19 | 50.88 | 47.53 |
| | DC | 92.44 | 38.28 | 41.37 | 38.24 |
| | AC | 20.58 | 8.91 | 9.51 | 9.29 |
| | PI = AC/DC | 22.3% | 23.3% | 23.0% | 24.3% |

The above-mentioned embodiments are provided for illustrative purposes to explain the principles and advantages of the present application and are not restrict the scope of the present application. Anyone skilled in the relevant technical field of the present application may make modifications and variations to the above embodiments without departing from the technical principles and spirit of the present application. All such modifications and variations that fall within the scope of the patent claims filed with this application, in terms of shapes, structures, features, and principles disclosed in this application, should be considered as being within the scope of the present application.

What is claimed is:

1. A detection element of biological subcutaneous features, comprising:
a substrate;
a light-detecting semiconductor chip is located on the substrate and is configured to detect intensity of red light or near-infrared light;
a grid structure is located on the light-detecting semiconductor chip; and
a cover is located between the grid structure and on the light-detecting semiconductor chip to protect a light incident surface of the detection element.

2. The detection element of biological subcutaneous features as claimed in claim 1, wherein the light-detecting semiconductor chip is made of silicon-based semiconductor material or III-V semiconductor material.

3. The detection element of biological subcutaneous features as claimed in claim 1, wherein the grid structure is configured to block side light and increase a proportion of near-vertical incident light.

4. The detection element of biological subcutaneous features as claimed in claim 1, wherein the grid structure comprises a plurality of opaque blocking walls that are parallel to each other, wherein an aspect ratio of the opaque blocking walls ranges from 1:1 to 100:1.

5. The detection element of biological subcutaneous features as claimed in claim 1, wherein the grid structure is composed of a plurality of opaque blocking walls that are parallel to each other, wherein a height of the opaque blocking walls ranges from 100 μm to 1000 μm, a width of the opaque blocking walls ranges from 30 μm to 100 μm, and a spacing of the opaque blocking walls ranges from 50 μm to 600 μm.

6. The detection element of biological subcutaneous features as claimed in claim 1, wherein the grid structure is formed directly on the light-detecting semiconductor chip using molding, cutting or etching.

7. The detection element of biological subcutaneous features as claimed in claim 6, wherein the grid structure is produced separately and attached onto the light-detecting semiconductor chip using an adhesive material, or affixed onto the light-detecting semiconductor chip with a clasp.

8. The detection element of biological subcutaneous features as claimed in claim 1, wherein the grid structure is made of opaque materials such as black resin, black silicone, and silicon substrate.

9. The detection element of biological subcutaneous features as claimed in claim 1, wherein the plurality of opaque blocking walls that are parallel to each other of the grid structure have an arrangement design with equal spacing.

10. The detection element of biological subcutaneous features as claimed in claim 1, wherein the plurality of opaque blocking walls that are parallel to each other of the grid structure have an arrangement with gradually sparse spacing.

11. The detection element of biological subcutaneous features as claimed in claim 1, wherein the plurality of opaque blocking walls that are parallel to each other of the grid structure have an arrangement with gradually dense spacing.

12. The detection element of biological subcutaneous features as claimed in claim 1, wherein the cover is made of materials that are substantially transparent to red light or near-infrared light, such as transparent resin, transparent silicone, or transparent glass.

13. A wearable device for detecting biological subcutaneous features, comprising:
a light source of red light or near-infrared light, and
a plurality of detection elements of biological subcutaneous features;
wherein at least one detection element of biological subcutaneous features has a grid structure, the grid structure has a plurality of opaque blocking walls that are parallel to each other, an arrangement direction of the opaque blocking walls is generally parallel to an emission direction of light emitted by the light source.

14. The wearable device for detecting biological subcutaneous features as claimed in claim 13, wherein the plurality of detection elements of biological subcutaneous features are arranged around the light source of red light or near-infrared light.

15. A wearable device for detecting biological subcutaneous features, comprising:
a light source of red light and near-infrared light, and
a plurality of detection elements of biological subcutaneous features;
wherein at least one detection element of biological subcutaneous features has a grid structure, the grid structure has a plurality of opaque blocking walls that are parallel to each other, an arrangement direction of the opaque blocking walls is generally parallel to an emission direction of light emitted by the light source.

16. The wearable device for detecting biological subcutaneous features as claimed in claim 15, wherein the plurality of detection elements of biological subcutaneous features are arranged around the light source of red light and near-infrared light.

17. A wearable device for detecting biological subcutaneous features, comprising:
a light source of red light or near-infrared light, and
a plurality of detection elements of biological subcutaneous features as claim 1.

18. The wearable device for detecting biological subcutaneous features as claimed in claim 17, wherein the plurality of detection elements of biological subcutaneous features are arranged around the light source of red light or near-infrared light.

19. A wearable device for detecting biological subcutaneous features, comprising:
a light source of red light and near-infrared light, and
a plurality of detection elements of biological subcutaneous features as claim 1.

20. The wearable device for detecting biological subcutaneous features as claimed in claim 19, wherein the plurality of detection elements of biological subcutaneous features are arranged around the light source of red light and near-infrared light.
